(19) United States
(12) Patent Application Publication
(10) Pub. No.: US 2017/0007651 A1
(43) Pub. Date: Jan. 12, 2017

(54) Energy Composition

(71) Applicants: Jesse McMullin, Riverton, UT (US); Mark Clark, South Jordan, UT (US)

(72) Inventors: Jesse McMullin, Riverton, UT (US); Mark Clark, South Jordan, UT (US)

(21) Appl. No.: 15/206,009

(22) Filed: Jul. 8, 2016

Related U.S. Application Data
(60) Provisional application No. 62/190,585, filed on Jul. 9, 2015.

(51) Int. Cl. A61K 36/17; A61K 31/194; A61K 31/475; A61K 36/185; A61K 31/522; A61K 31/137

(52) U.S. Cl. CPC A61K 36/17; A61K 31/522; A61K 31/137; A61K 31/475; A61K 36/185; A61K 31/194

(57) ABSTRACT

Some embodiments of the invention include an energy composition. In some embodiments, the energy composition may include an herb such as Mormon tea, a methylxanthine such as theobromine, and an active ingredient such as phenylethylamine HCl.

### FIELD

The embodiments discussed herein are related to an energy composition.

### SUMMARY

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

Some example embodiments generally relate to an energy composition configured to produce an energy boost or other effect and/or a method of formulating the energy composition. In some embodiments, an energy composition may comprise of one or more of the following: an herb including Mormon tea, a methylxanthine including theobromine, and/or an active ingredient including phenylethylamine HCl. In some embodiments, an energy composition may reduce the delay between consumption and effect of the energy composition, produce an energy boost or other effect, and/or stabilize the perceived increase of energy or effect, reducing the potential for a “sugar crash” or a “caffeine crash.”

In some embodiments, the herb may include plants or extracts from plants that produce an energy boost or effect of an energy composition. In various embodiments, the herb may include, for example, Mormon tea, guayusa, and/or chaparro prieto. In some embodiments, the herb may be brewed, which may reduce the delay between consumption and effect of the energy and/or may increase the effect of the energy composition. In some embodiments, a stainless steel mesh basket may be used to brew the tea for a minimum of 30 minutes and/or until the active constituents are released.

In some embodiments, the methylxanthine may include caffeine or theobromine. In various embodiments, the methylxanthine may include caffeine and theobromine, which may stabilize the effects of the energy composition, reducing the potential for a “sugar crash” or a “caffeine crash.”

In some embodiments, the active ingredient may include phenylethylamine HCl. In various embodiments, the active ingredient may also include at least one of the following: glucuronolactone, malic acid, taurine, and/or yohimbine HCl.

In some embodiments, a method of formulating an energy composition may comprise, for example, heating water to a temperature from about 80 to 100 degrees Celsius, adding Mormon tea in an amount from about 0.000082 g/mL to about 0.0047 g/mL to the water, creating an herb and water mixture. The herb and water mixture may be, for example, removed from the heat and soaked for no shorter than 30 minutes or until active constituents are released. The herb and water mixture may be, for example, mixed with a methylxanthine including theobromine in an amount from about 0.000083 g/mL to about 0.0012 g/mL and mixed with an active ingredient including phenylethylamine HCl in an amount from about 0.00094 g/mL to about 0.010 g/mL. The energy composition may, for example, be formulated in a predetermined volume for consumption.

The method of formulating an energy composition may include, for example, adding caffeine in an amount from about 0.0028 g/mL to about 0.0050 g/mL. The method of formulating an energy composition may include, for example, adding caffeine and theobromine in an amount from about 0.0028 g/mL to about 0.0062 g/mL, coupled to stabilize the perceived effects. The method of formulating an energy composition may include, for example, adding malic acid in an amount from about 0.010 g/mL to about 0.014 g/mL. The method of formulating an energy composition may include, for example, adding yohimbine HCl in an amount from about 0.000049 g/mL to about 0.000098 g/mL.

In some embodiments, an energy composition may comprise, for example, Mormon tea in an amount from about 0.00037 g/mL to about 0.0015 g/mL, theobromine and caffeine in an amount from about 0.0031 g/mL to about 0.0039 g/mL, and phenylethylamine HCl in an amount from about 0.0018 g/mL to about 0.0042 g/mL.

The energy composition may further comprise, for example, water, wherein the Mormon tea is brewed in the water for no shorter than 30 minutes or until active constituents are released. The energy composition may comprise, for example, caffeine in an amount from about 0.0030 g/mL to about 0.0038 g/mL. The energy composition may comprise, for example, malic acid in an amount from about 0.010 g/mL to about 0.014 g/mL. The energy composition may comprise, for example, yohimbine HCl in an amount from about 0.000049 g/mL to about 0.000097 g/mL.

In some embodiments, an energy composition may comprise, for example, an herb including Mormon tea in an amount from about 0.000082 g/mL to about 0.0047 g/mL, a methylxanthine including theobromine in an amount from about 0.000083 g/mL to about 0.0012 g/mL, and an active ingredient including phenylethylamine HCl in an amount from about 0.00094 g/mL to about 0.0083 g/mL.

The energy composition may further comprise, for example, water, wherein the Mormon tea is brewed in the water for no shorter than 30 minutes or until active constituents are released. The energy composition may further comprise, for example, an herb including guayusa in an amount from about 0.00041 g/mL to about 0.0012 g/mL. The energy composition may further comprise, for example, a methylxanthine including caffeine in an amount from about 0.0028 g/mL to about 0.0050 g/mL. The energy composition may comprise, for example, caffeine and theobromine in an amount from about 0.0028 g/mL to about 0.0062 g/mL, coupled to stabilize the perceived effects. The energy composition may comprise, for example, an active ingredient including malic acid in an amount from about 0.011 g/mL to about 0.014 g/mL. The energy composition may comprise, for example, an active ingredient including yohimbine HCl in an amount from about 0.000066 g/mL to about 0.000083 g/mL.

In some embodiments, an energy composition may comprise, for example, Mormon tea in an amount from about 0.000018 g/mL to about 0.019 g/mL, a methylxanthine including theobromine in an amount from about 0.000060 g/mL to about 0.0019 g/mL and caffeine in an amount from about 0.00083 g/mL to about 0.012 g/mL, and an active ingredient including phenylethylamine HCl in an amount from about 0.000018 g/mL to about 0.010 g/mL.

The energy composition may further comprise, for example, water, wherein the Mormon tea is brewed in the water for no shorter than 30 minutes or until active constituents are released. The energy composition may comprise, for example, an active ingredient including malic acid in an amount from about 0.0083 g/mL to about 0.034 g/mL. The energy composition may comprise, for example, an active ingredient including yohimbine HCl in an amount from about 0.000015 g/mL to about 0.00017 g/mL.

Additional features and advantages of the invention will be set forth in the Detailed Description which follows, and, in part, will be obvious from the description, or may be learned by the practice of the invention. The features and advantages of the invention may be realized and obtained by means of the combinations particularly pointed out in the appended claims. These and other features of the present invention will become more fully apparent from the following description and appended claims, or may be learned by the practice of the invention as set forth hereinafter.

### BRIEF DESCRIPTION OF THE DRAWINGS

To further clarify the above and other advantages of the present invention, a more particular description of the invention will be rendered by reference to example embodiments thereof which are illustrated in a graph and a block diagram. Example embodiments of the invention will be descripted and explained with additional specificity and detail through the use of the accompanying drawings. FIGS. 1 and 2 depict only example embodiments and are not to be considered limiting of scope.

FIG. 1 is a graph that illustrates the perceived effect of some ingredients of an energy composition according to some embodiments.

FIG. 2 illustrates a block diagram of an example method for producing an energy composition according to some embodiments.

### DETAILED DESCRIPTION

Energy compositions are often consumed to experience an energy boost, increased alertness, accelerated heart rate, and/or an increased metabolism (“perceived effects”). The perceived effects of consuming an energy composition also may include a variety of perceived physical changes such as, for example, a euphoric high, increased alertness, increased focus, increased metabolism, increased heart rate, and/or any other enhanced physical change. Energy compositions may be consumed in situations where any of the perceived effects may be beneficial. Such situations may include, for example, in the morning, late at night, and/or extended periods of driving. Energy compositions have also become popular at clubs and bars, where the perceived effects may be associated with a euphoric feeling. Energy compositions may refer to any other edible or absorbable compositions such as, for example, an edible liquid, an edible powder, an edible solid, a liposomal cream, and/or any other composition suitable for delivering compositions to the body.

A possible problem associated with many commonly used energy compositions, for example, may be a perceived delay between consumption of the energy composition and the perceived effects. Another possible problem associated with many commonly used energy compositions, for example, may be the drastic reduction of the perceived effects as the energy composition wears off (“caffeine crash” and/or “sugar crash”). For example, some commonly used energy compositions' perceived effects have a minimal lasting effect. Also, physical shaking, or jitters, and sleep disruptions may be possible problems associated with many commonly used energy compositions. Additionally, for example, many commonly used energy compositions' perceived effects may have minimal strength.

Some embodiments of the present invention include an energy composition. In some embodiments, the energy composition may be configured in a manner that provides one or more of the following benefits: reduce the delay between consumption and the perceived effects of the energy composition, increase the strength of the perceived effects, increase the length of time the perceived effects are experienced, and/or stabilize the reduction of the perceived effects to reduce the potential for a “sugar crash” or a “caffeine crash.”

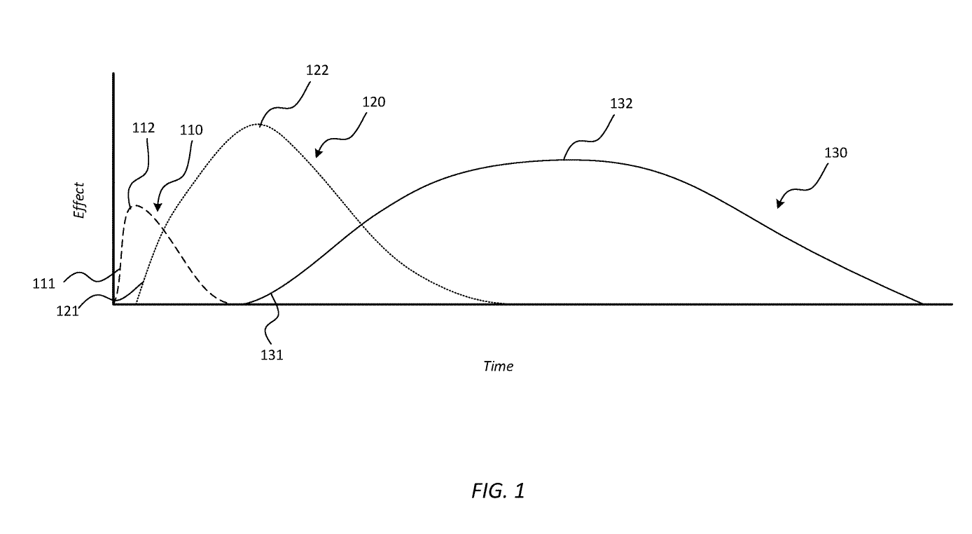

In some embodiments, the use of Mormon tea, caffeine, theobromine, and/or phenylethylamine HCl in combination may provide one or more of the following benefits: reduce the delay between consumption and the perceived effects, increase the strength of the perceived effects, increase the length of time the perceived effects are experienced, and stabilize the reduction of the perceived effects to reduce the potential for a “sugar crash” or a “caffeine crash.” FIG. 1 is a graph of one or more perceived effects over time from ingestion of the energy composition as described in accordance with some embodiments.

One or more perceived effects of Mormon tea, for example, may take effect about 5 to 10 minutes after ingestion of the energy composition. The half-life of Mormon tea, for example, is about 15 to 20 minutes. Phenylethylamine HCl, for example, may react with Mormon tea to substantially produce one or more perceived effects between 5 minutes and 25 minutes after consumption of the energy composition. One or more perceived effects of caffeine, for example, may take effect about 30 to 40 minutes after ingestion of the energy composition. The half-life of caffeine, for example, is about 2 to 5 hours. One or more perceived effects of theobromine, for example, may take effect about 2 to 3 hours after ingestion. The half-life of theobromine, for example, is about 7 to 12 hours.

FIG. 1 is a graph showing the onset, half-life, and dissipation of some ingredients of an example energy composition as described in accordance with some embodiments. The x-axis of FIG. 1 represents time. The y-axis of FIG. 1 represents the change in perceived effects of some ingredients of an example energy composition. The graph shown in FIG. 1 is not drawn to scale. Moreover, the various curves may be have higher or lower maximums that what is shown in FIG. 1 and/or the duration of a curve may be extended or shrunk. FIG. 1 depicts only one example embodiment and is not intended to be limiting in scope. The various curves in the graph shown in FIG. 1 may not be drawn to scale. For example, line 111 shows the perceived effects of Mormon tea over time. The line 111 shows the perceived effects of Mormon tea may begin to take effect, for example, about 5 minutes after ingesting the energy composition. The half-life of Mormon tea is shown as peak 112 in the line 111, which, for example, may be about 20 minutes after ingestion of the energy composition. Dissipation of the perceived effects of Mormon tea is shown in the tail 110 of the line 111. As the perceived effects of Mormon tea begin to dissipate or dissipate (see tail 110 of line 111), the perceived effects of caffeine may begin to take effect as shown by line 121 representing the perceived effects of caffeine over time. The perceived effects of caffeine may begin to take effect, for example, about 30 minutes after ingestion of the energy composition. The half-life of caffeine is shown as peak 122 in the line 121, which, for example, may be about 2.5 hours after ingestion of the energy composition. Dissipation of the perceived effects of caffeine is shown in the tail 120 of line the 112. The perceived effects of caffeine may rapidly dissipate. As the perceived effects of caffeine begin to dissipate or dissipate (see tail 120 of line 121), the perceived effects of theobromine may begin to take effect as shown by line 131 representing the perceived effects of theobromine over time. The perceived effects of theobromine may begin to take effect, for example, about 2 hours after ingestion of the energy composition. The half-life of theobromine is shown as peak 132 in the line 131, which, for example, may be about 7 hours after ingestion of the energy composition.

Although the perceived effects of caffeine may not take a long time to take effect, the addition of Mormon tea, for example, may reduce the delay between consumption and the perceived effects. Additionally, the perceived effects of Mormon tea may be increasingly strong proportional to the amount of time Mormon tea is brewed. After the half-life of the caffeine is reached and/or the perceived effects of the caffeine begin to dissipate, people generally may feel a “caffeine crash” because the perceived effects, for example, may wear off rapidly. However, the addition of theobromine, for example, may stabilize the reduction of the perceived effects to reduce the potential for a “sugar crash” or “caffeine crash.” Additionally, the perceived effects of theobromine, for example, although more potent, may be significantly slower than caffeine, which may prevent the perceived effects from wearing off rapidly. Theobromine's slow onset and long-lasting perceived effects, for example, may counter caffeine's rapid onset and dissipation. The addition of theobromine, for example, may increase the length of time the perceived effects are experienced and/or reduce the potential for a “sugar crash” or “caffeine crash.”

In some embodiments, the energy composition, for example, may include a delivery system, an herb including Mormon tea, a methylxanthine including theobromine, and an active ingredient including phenylethylamine HCl. The delivery system, for example, may be configured to deliver the active ingredient in an edible composition. For example, the medium may include edible solids, edible liquids, edible powders, liposomal creams, and/or any other delivery system configured to transfer chemicals into a body.

For example, edible solids may include: hard candy; soft candy; chewable candy; liquid capsulated pills; soft gel pills; chewable pills; multi-layered pills; dissolvable pills; effervescent pills configured to dissolve in liquids; lozenges; “gummy” candy; gelatin; edible food bars; dissolvable film; chewing gum; mints; frosting; edible coatings; frozen edible liquids; lip balm; and/or any other solid suitable for consumption. Embodiments including multi-layered pills, for example, may be configured such that each layer delivers a separate component of the energy composition.

For example, edible liquids may include: water; juice, including juice in concentrate; shakes; smoothies; edible gels; pastes, including semi-edible pastes such as toothpaste; mouthwash; vapors, such as, for example, humidifiers, aerosol sprays, breath sprays, nasal sprays, inhalers, electronic cigarette, and/or any other vapor suitable for inhalation; eye drops; condiments, such as sauces, syrup, honey, whipped cream and/or other condiment; and/or any other liquid or semi-liquid suitable for consumption. In some embodiments, the edible liquid may be frozen, for example, as an ice cube.

For example, edible powders may include: granulated edible solids; mixable powder configured to substantially dissolve into an edible liquid; powder configured to substantially mix with baking ingredients, such as, for example, baking powder, and/or sweetener; powder configured to be inhaled through the nasal cavity; edible candy powder; and/or any other edible powder suitable for consumption or substantial mixing with an edible liquid.

For example, liposomal creams may include: lotions; cosmetics; cosmetic creams; face wash; and/or any other liposomal cream suitable for substantial dermal absorption of chemicals.

The delivery system is not limited to only one category of delivery systems specified. In some embodiments, the delivery system, for example, may be composed of any number or combination of delivery systems, such as, for example, a mixable powder and edible liquid.

The herb may include plants or extracts from plants. Such herbs may include, for example, Mormon tea, guayusa, and/or chaparro prieto. Herbs that include teas are not expressly limited to use with water, but may also include, for example, waterless compositions such as powders, leaves, and/or any other compositions that do not substantially use water. In some embodiments including chaparro prieto, chaparro prieto may be included, for example, in amounts from about 0.000018 g/mL to about 0.019 g/mL; and/or in amounts from about 0.00046 g/mL to about 0.0014 g/mL; and/or in amounts from about 0.0015 g/mL to about 0.0024 g/mL; and/or in amounts from about 0.0028 g/mL to about 0.0047 g/mL; and/or any other range suitable for use in an energy composition. In some embodiments including Mormon tea, Mormon tea may be included, for example, in amounts from about 0.000018 g/mL to about 0.019 g/mL; and/or in amounts from about 0.00037 g/mL to about 0.0015 g/mL; and/or in amounts from about 0.0015 g/mL to about 0.0024 g/mL; and/or in amounts from about 0.0025 g/mL to about 0.0037 g/mL; and/or in amounts from about 0.0038 g/mL to about 0.0047 g/mL; and/or in amounts from about 0.000082 g/mL to about 0.0047 g/mL; and/or any other range suitable for use in an energy composition. In some embodiments including guayusa, guayusa may be included, for example, in amounts from about 0.00018 g/mL to about 0.012 g/mL; and/or in amounts from about 0.0016 g/mL to about 0.0051 g/mL; and/or in amounts from about 0.0030 g/mL to about 0.0038 g/mL; and/or any other range suitable for use in an energy composition.

In some embodiments, the methylxanthine may include, for example, theobromine or caffeine. In some embodiments, the methylxanthine may include, for example, theobromine and caffeine. In some embodiments, theobromine may be included, for example, in amounts from about 0.000060 g/mL to about 0.012 g/mL; and/or in amounts from about 0.000094 g/mL to about 0.00028 g/mL; and/or in amounts from about 0.00030 g/mL to about 0.00094 g/mL; and/or in amounts from about 0.0010 g/mL to about 0.0019 g/mL; and/or in amounts from about 0.00083 g/mL to about 0.0012 g/mL; and/or in amounts from about 0.000060 g/mL to about 0.0019 g/mL; and/or any other range suitable for use in an energy composition.

In some embodiments, caffeine may be included, for example, in amounts from about 0.000018 g/mL to about 0.012 g/mL; and/or in amounts from about 0.00083 g/mL to about 0.012 g/mL; and/or in amounts from about 0.0028 g/mL to about 0.0050 g/mL; and/or in amounts from about 0.0030 g/mL to about 0.0038 g/mL; and/or in amounts from about 0.0035 g/mL to about 0.0057 g/mL; and/or any other range suitable for use in an energy composition.

Some embodiments may include, for example, one quantity of a methylxanthine substitute for another quantity of a separate methylxanthine. Some embodiments may include, for example, any combination of different quantities of methylxanthine. In some embodiments including theobromine and caffeine, caffeine and theobromine may be included, for example, in amounts from about 0.0031 g/mL to about 0.0039 g/mL; and/or in amounts from about 0.0028 g/mL to about 0.0062 g/mL; and/or in amounts from about 0.00089 g/mL to about 0.014 g/mL; and/or any other suitable range for use in an energy composition.

The active ingredient may include, for example, phenylethylamine HCl. In some embodiments, the active ingredient may also include one or more of the following: glucuronolactone, malic acid, taurine, yohimbine, HCl, and/or any other ingredient configured in a manner that may produce the perceived effects. In some embodiments, the active ingredient may be included in any amount suitable for use in an energy composition.

In some embodiments including phenylethylamine HCl, phenylethylamine HCl may be included, for example, in amounts from about 0.000018 g/mL to about 0.010 g/mL; and/or in amounts from about 0.00094 g/mL to about 0.0018 g/mL; and/or in amounts from about 0.0018 g/mL to about 0.0042 g/mL; and/or in amounts from about 0.00094 g/mL to about 0.0083 g/mL; and/or in amounts from about 0.0030 g/mL to about 0.0094 g/mL; and/or any other range suitable for use in an energy composition.

In some embodiments including malic acid, malic acid may be included, for example, in amounts from about 0.00018 g/mL to about 0.034 g/mL; and/or in amounts from about 0.0094 g/mL to about 0.014 g/mL; and/or in amounts from about 0.0083 g/mL to about 0.034 g/mL; and/or in amounts from about 0.013 g/mL to about 0.016 g/mL; and/or in amounts from about 0.015 g/mL to about 0.038 g/mL; and/or any other range suitable for use in an energy composition.

In some embodiments including yohimbine HCl, yohimbine HCl may be included, for example, in amounts from about 0.000015 g/mL to about 0.00019 g/mL; and/or in amounts from about 0.000049 g/mL to about 0.00011 g/mL; and/or in amounts from about 0.000066 g/mL to about 0.000083 g/mL; and/or any other range suitable for use in an energy composition.

In some embodiments including glucuronolactone, glucuronolactone may be included, for example, in amounts from about 0.00018 g/mL to about 0.0076 g/mL; and/or in amounts from about 0.0033 g/mL to about 0.0042 g/mL; and/or in amounts from about 0.0018 g/mL to about 0.0057 g/mL; and/or any other range suitable for use in an energy composition.

In some embodiments including taurine, taurine may be included, for example, in amounts from about 0.00018 g/mL to about 0.029 g/mL; and/or in amounts from about 0.0094 g/mL to about 0.012 g/mL; and/or in amounts from about 0.013 g/mL to about 0.016 g/mL; and/or in amounts from about 0.015 g/mL to about 0.038 g/mL; and/or any other range suitable for use in an energy composition.

Some embodiments also may include, for example, one or more flavorants that enhance the palatability of the energy composition. Such flavorants may include, for example, acesulfame potassium, artificial strawberry, bitter blocker, citric acid, grape, N&A kiwi, N&A lemon-lime, N&A watermelon, natural bitter blocker, natural kiwi, natural strawberry, sodium chloride, sucralose, and/or any other flavorant suitable for improving palatability. One or more flavorants may be included in any number of quantities or combinations suitable for improving palatability.

Some embodiments also may include, for example, one or more preservatives configured to improve shelf life of the energy composition. Preservatives may include, for example, potassium sorbate, sodium benzoate, or any other preservative suitable for improving shelf life of an energy composition. The preservatives may be included in any combination or quantity suitable for improving the shelf life of the energy composition.

Some embodiments also may include, for example, one or more coloring agents configured to alter the color of the energy composition. Coloring agents may include, for example, blue lake #1, red lake #40, or any other coloring agent suitable for use in an energy composition. Coloring agents may be included in any quantity or combination configured to alter the color of the energy composition.

Some embodiments may include, for example, a gumming agent configured to provide a palatable texture, consistency, and/or enhance absorption of at least one flavorant. The gumming agent may include, for example, xanthan gam, cellulose gum, carrageenan gum, or any other gum suitable for use in an energy composition. In embodiments including a gumming agent, the gumming agent may be included, for example, in amounts from about 0.00018 g/mL to about 0.012 g/mL; or in amounts from about 0.00022 g/mL to about 0.00045 g/mL; or in amounts from about 0.00050 g/mL to about 0.00085 g/mL; or any other range suitable for use in an energy composition.

Some embodiments of the energy composition may include, for example, a delivery system and a combination of at least one herb, at least one methylxanthine, and at least one active ingredient. One or more herbs, one or more forms of methylxanthine, and one or more active ingredients may be combined in a manner suitable for producing the perceived effects of the energy composition. Such combinations may include, for example, any of the previously specified ranges of any of the herbs, forms of methylxanthine, and active ingredients. Additionally, such embodiments may include, for example, a gumming agent and one or more flavorants.

In some embodiments, a method of formulating an energy composition may comprise, for example, heating water to a temperature from about 80 to 100 degrees Celsius, adding Mormon tea in an amount from about 0.000082 g/mL to about 0.0047 g/mL to the water, creating an herb and water mixture. The herb and water mixture may be, for example, removed from the heat and soaked for no shorter than 30 minutes or until active constituents are released. The herb and water mixture may be, for example, mixed with a methylxanthine including theobromine in an amount from about 0.000083 g/mL to about 0.0012 g/mL and mixed with an active ingredient including phenylethylamine HCl in an amount from about 0.00094 g/mL to about 0.010 g/mL. The energy composition may, for example, be formulated in a predetermined volume for consumption.

The method of formulating an energy composition may include, for example, adding caffeine in an amount from about 0.0028 g/mL to about 0.0050 g/mL. The method of formulating an energy composition may include, for example, adding caffeine and theobromine in an amount from about 0.0028 g/mL to about 0.0062 g/mL, coupled to stabilize the perceived effects. The method of formulating an energy composition may include, for example, adding malic acid in an amount from about 0.010 g/mL to about 0.014 g/mL. The method of formulating an energy composition may include, for example, adding yohimbine HCl in an amount from about 0.000049 g/mL to about 0.000098 g/mL.

In some embodiments, an energy composition may comprise, for example, Mormon tea in an amount from about 0.00037 g/mL to about 0.0015 g/mL, theobromine and caffeine in an amount from about 0.0031 g/mL to about 0.0039 g/mL, and phenylethylamine HCl in an amount from about 0.0018 g/mL to about 0.0042 g/mL.

The energy composition may further comprise, for example, water, wherein the Mormon tea is brewed in the water for no shorter than 30 minutes or until active constituents are released. The energy composition may comprise, for example, caffeine in an amount from about 0.0030 g/mL to about 0.0038 g/mL. The energy composition may comprise, for example, malic acid in an amount from about 0.010 g/mL to about 0.014 g/mL. The energy composition may comprise, for example, yohimbine HCl in an amount from about 0.000049 g/mL to about 0.000097 g/mL.

In some embodiments, an energy composition may comprise, for example, an herb including Mormon tea in an amount from about 0.000082 g/mL to about 0.0047 g/mL, a methylxanthine including theobromine in an amount from about 0.000083 g/mL to about 0.0012 g/mL, and an active ingredient including phenylethylamine HCl in an amount from about 0.00094 g/mL to about 0.0083 g/mL.

The energy composition may further comprise, for example, water, wherein the Mormon tea is brewed in the water for no shorter than 30 minutes or until active constituents are released. The energy composition may further comprise, for example, an herb including guayusa in an amount from about 0.00041 g/mL to about 0.0012 g/mL. The energy composition may further comprise, for example, a methylxanthine including caffeine in an amount from about 0.0028 g/mL to about 0.0050 g/mL. The energy composition may comprise, for example, caffeine and theobromine in an amount from about 0.0028 g/mL to about 0.0062 g/mL, coupled to stabilize the perceived effects. The energy composition may comprise, for example, an active ingredient including malic acid in an amount from about 0.011 g/mL to about 0.014 g/mL. The energy composition may comprise, for example, an active ingredient including yohimbine HCl in an amount from about 0.000066 g/mL to about 0.000083 g/mL.

In some embodiments, an energy composition may comprise, for example, Mormon tea in an amount from about 0.000018 g/mL to about 0.019 g/mL, a methylxanthine including theobromine in an amount from about 0.000060 g/mL to about 0.0019 g/mL and caffeine in an amount from about 0.00083 g/mL to about 0.012 g/mL, and an active ingredient including phenylethylamine HCl in an amount from about 0.000018 g/mL to about 0.010 g/mL.

The energy composition may further comprise, for example, water, wherein the Mormon tea is brewed in the water for no shorter than 30 minutes or until active constituents are released. The energy composition may comprise, for example, an active ingredient including malic acid in an amount from about 0.0083 g/mL to about 0.034 g/mL. The energy composition may comprise, for example, an active ingredient including yohimbine HCl in an amount from about 0.000015 g/mL to about 0.00017 g/mL.

The following tables provide a few examples of an energy composition according to some embodiments. In the examples provided in the following tables, the listed ingredients may be combined with the delivery system. In embodiments where the energy composition is an edible liquid, the delivery system may be configured as an edible liquid, such as, for example, water. In these embodiments, the sum of the listed ingredients and the edible liquid may have a total volume of about 53 mL.

In some embodiments, such as, for example, that illustrated in Table 1, the energy composition may include, for example, 25 mg to 75 mg of Mormon tea. Mormon tea may be, for example, added to water that is between 80 and 100 degrees Celsius. Mormon tea may be brewed in the heated water for no less than 30 minutes and/or until the active constituents are released. Mormon tea may produce the perceived effects almost immediately after ingestion, wherein Mormon tea may take effect, for example, in about 5 to 10 minutes and wherein the term “immediately” means between 5 and 25 minutes. The half-life of Mormon tea, for example, is about 15 to 20 minutes. The perceived effects of Mormon tea may be increasingly strong effect proportional to the amount of time Mormon tea is brewed.

In some embodiments, the energy composition may also include, for example, 190 mg to 210 mg of caffeine and 5 mg to 15 mg of theobromine. Caffeine may take effect, for example, about 30 to 40 minutes after ingestion, and caffeine has a half-life, for example, of about 2.5 to 5 hours. Theobromine may take effect, for example, about 2 to 3 hours after ingestion, and theobromine has a half-life, for example, of about 7 to 12 hours. The combination of caffeine and theobromine, for example, may create longer lasting perceived effects. The addition of both caffeine and theobromine, for example, may reduce the potential for a “sugar crash” or “caffeine crash.” For example, theobromine may take effect as the caffeine reaches its half-life. Additionally, the onset of the perceived effects of theobromine, although more potent, may be significantly slower than caffeine. Thus, the addition of theobromine may, for example, prevent a “sugar crash” or “caffeine crash.”

In some embodiments, the energy composition may also include, for example, 110 mg to 146 mg of phenylethylamine HCl. Phenylethylamine HCl may, for example, increase cognitive enhancement and mood improvement, and/or act as a concentration aid. Phenylethylamine HCl may, for example, react with Mormon tea in a manner that may create the perceived effects immediately. Phenylethylamine HCl may create the perceived effects of natural energy and heightened mental concentration.

In some embodiments, the energy composition may include, for example, 180 mg to 220 mg of glucuronolactone. The energy composition may also include, for example, 700 mg to 800 mg of malic acid. The energy composition may include, for example, 700 mg to 800 mg of taurine. The energy composition may include, for example 4 mg to 5 mg of yohimbine HCl.

In some embodiments, the energy composition may include, for example, any combination of herbs, methylxanthines, and active ingredients. Additionally, such embodiments may include a gumming agent and/or one or more flavorants. In some embodiment, the energy composition may include, for example, 27 mg to 45 mg of gumming agent and 1300 mg to 1400 mg of flavorants.

TABLE 1

| Component | Amount (mg) |
|---|---|
| Caffeine | 190-210 |
| Mormon tea | 25-75 |
| Glucuronolactone | 180-220 |
| Malic Acid | 700-800 |
| Phenylethylamine HCl | 110-146 |
| Taurine | 700-800 |
| Theobromine | 5-15 |
| Yohimbine HCl | 4-5 |
| Gumming Agent | 27-45 |
| Preservatives | 50-60 |
| Flavorants | 1300-1400 |
| Color | 15-25 |

In some embodiments, such as, for example, that illustrated in Table 2, the energy composition may include, for example, 35 mg to 85 mg of Mormon tea. Mormon tea takes effect, for example, in about 5 to 10 minutes. The half-life of Mormon tea, for example, is about 15 to 20 minutes. The perceived effects of Mormon tea may be increasingly strong proportional to the amount of time Mormon tea is brewed. Additionally, the perceived effects may be a stronger when the amount of Mormon tea is increased in the energy composition.

In some embodiments, the energy composition may also include, for example, 60 mg to 260 mg of caffeine and 10 mg to 50 mg of theobromine. Including theobromine may prevent a “sugar crash” or “caffeine crash.” For example, theobromine may take effect as caffeine reaches its half-life, which may reduce the potential for a “sugar crash” or “caffeine crash.” Theobromine's slow onset and long lasting perceived effects, for example, may counter caffeine's rapid onset and dissipation. Additionally, the perceived effects of theobromine may be mild in comparison to caffeine, which may create a slow taper as the perceived effects of theobromine wear off.

In some embodiments, the energy composition may also include, for example, 100 mg to 250 mg of phenylethylamine HCl. Phenylethylamine HCl, for example, may react with Mormon tea in a manner that may create the perceived effects immediately. Phenylethylamine HCl may create the perceived effects of natural energy and heightened mental concentration.

In some embodiments, the energy composition may include, for example, 100 mg to 300 mg of glucuronolactone. The energy composition may also include, for example, 340 mg to 1060 mg of malic acid. The energy composition may include, for example, 375 mg to 1170 mg of taurine. The energy composition may include, for example, 3 mg to 6 mg of yohimbine HCl.

In some embodiments, the energy composition may include, for example, any combination of herbs, methylxanthines, and active ingredients. Additionally, such embodiments may include a gumming agent and/or one or more flavorants. In some embodiments, the energy composition may include, for example, 27 mg to 45 mg of gumming agent and 1300 mg to 1400 mg of flavorants.

TABLE 2

| Component | Amount (mg) |
|---|---|
| Caffeine | 60-260 |
| Mormon tea | 35-85 |
| Glucuronolactone | 100-300 |
| Malic Acid | 340-1060 |
| Phenylethylamine HCl | 100-250 |
| Taurine | 375-1170 |
| Theobromine | 10-50 |
| Yohimbine HCl | 3-6 |
| Gumming Agent | 27-45 |
| Preservatives | 50-60 |
| Flavorants | 1300-1400 |
| Color | 15-25 |

In some embodiments, such as, for example, that illustrated in Table 3, the energy composition may include, for example, 5 mg to 250 mg of Mormon tea. Mormon tea may be, for example, added to water that is between 80 and 100 degrees Celsius. Mormon tea may be brewed in the heated water for no less than 30 minutes and/or until the active constituents are released. Mormon tea may take effect, for example, in about 5 to 10 minutes. The half-life of Mormon tea, for example, is about 15 to 20 minutes. The perceived effects of Mormon tea may be increasingly strong proportional to the amount of time Mormon tea is brewed. Additionally, the perceived effects may be stronger when the amount of Mormon tea is increased in the energy composition.

In some embodiments, the energy composition may include, for example, 150 mg to 300 mg of caffeine and 5 mg to 100 mg of theobromine. The combination of caffeine and theobromine, for example, may create longer lasting perceived effects. The addition of both caffeine and theobromine, for example, may reduce the potential for a “sugar crash” or “caffeine crash.” Theobromine takes effect as the caffeine reaches its half-life. Additionally, the perceived effects of theobromine, although more potent, are significantly slower than caffeine. Thus, the addition of theobromine may prevent a “sugar crash” or “caffeine crash.”

In some embodiments, the energy composition may also include, for example, 80 mg to 500 mg of phenylethylamine HCl. Phenylethylamine HCl, for example, may react with Mormon tea in a manner that may create the perceived effects immediately. Phenylethylamine HCl may create the perceived effects of natural energy and heightened mental concentration.

In some embodiments, the energy composition may include, for example, 100 mg to 300 mg of glucuronolactone. The energy composition may also include, for example, 500 mg to 2000 mg of malic acid. The energy composition may include, for example, 500 mg to 2000 mg of taurine. The energy composition may include, for example 1 mg to 10 mg of yohimbine HCl.

In some embodiments, the energy composition may include, for example, any combination of herbs, methylxanthines, and active ingredients. Additionally, such embodiments may include a gumming agent and/or one or more flavorants. In some embodiments, the energy composition may include, for example, 10 mg to 600 mg of gumming agent and 750 mg to 2700 mg of flavorants.

TABLE 3

| Component | Amount (mg) |
|---|---|
| Caffeine | 150-300 |
| Mormon tea | 5-250 |
| Glucuronolactone | 100-300 |
| Malic Acid | 500-2000 |
| Phenylethylamine HCl | 80-500 |
| Taurine | 500-2000 |
| Theobromine | 5-100 |
| Yohimbine HCl | 1-10 |
| Gumming Agent | 10-600 |
| Preservatives | 50-70 |
| Flavorants | 750-2700 |
| Color | 5-25 |

In some embodiments, such as, for example, that illustrated in Table 4, the energy composition may include 1 mg to 1000 mg of Mormon tea. Mormon tea may take effect, for example, about 5 to 10 minutes after ingestion. The half-life of Mormon tea, for example, is about 15 to 20 minutes. The perceived effects of Mormon tea may be increasingly strong proportional to the amount of time Mormon tea is brewed. Additionally, the perceived effects may be stronger when the amount of Mormon tea is increased in the energy composition.

In some embodiments, the energy composition may also include, for example, 10 mg to 600 mg of caffeine and 10 mg to 600 mg of theobromine. Including theobromine may reduce the potential for a “sugar crash” or “caffeine crash.” For example, theobromine may take effect as caffeine reaches its half-life, which may reduce the potential for a “sugar crash” or “caffeine crash.” Theobromine's slow onset and long lasting perceived effects, for example, may counter caffeine's rapid onset and dissipation. Additionally, the perceived effects of theobromine may be mild in comparison to caffeine, which may create a slow taper as the perceived effects of theobromine wear off.

In some embodiments, the energy composition may also include, for example, 1 mg to 500 mg of phenylethylamine HCl. Phenylethylamine HCl, for example, may react with Mormon tea in a manner that may create the perceived effects immediately. Phenylethylamine HCl may create the effects of natural energy and heightened mental concentration.

In some embodiments, the energy composition may include, for example, 10 mg to 400 mg of glucuronolactone. The energy composition may also include, for example, 10 mg to 1500 mg of malic acid. The energy composition may include, for example, 10 mg to 1500 mg of taurine. The energy composition may include, for example 0.05 mg to 15 mg of yohimbine HCl.

In some embodiments, the energy composition may include, for example, any combination of herbs, methylxanthines, and active ingredients. Additionally, such embodiments may include a gumming agent and/or one or more flavorants. In some embodiments, the energy composition may include, for example, 10 mg to 600 mg of gumming agent and 750 mg to 2700 mg of flavorants.

TABLE 4

| Component | Amount (mg) |
|---|---|
| Caffeine | 10-600 |
| Mormon tea | 1-1000 |
| Guayusa | 10-600 |
| Glucuronolactone | 10-400 |
| Malic Acid | 10-1500 |
| Phenylethylamine HCl | 1-500 |
| Taurine | 10-1500 |
| Theobromine | 10-600 |
| Yohimbine HCl | 0.05-15 |
| Gumming Agent | 10-600 |
| Preservatives | 50-70 |
| Flavorants | 750-2700 |
| Color | 5-25 |

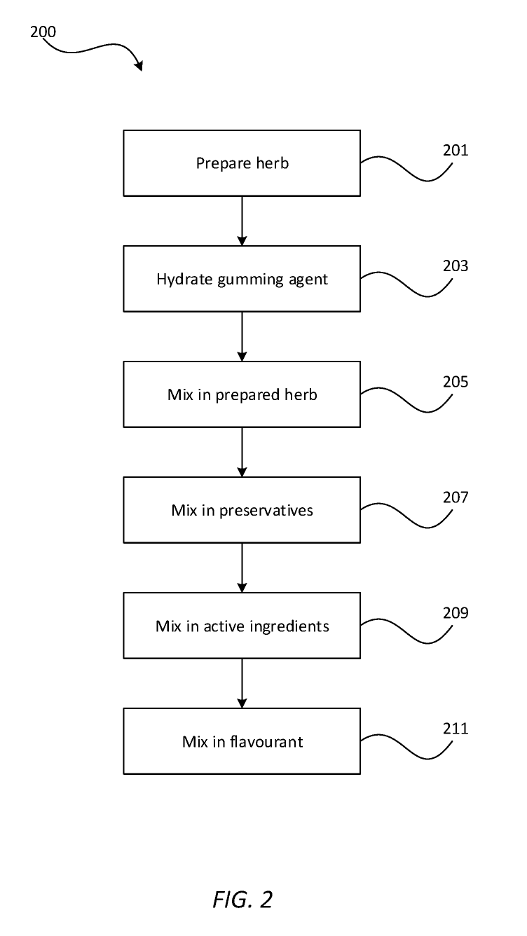

FIG. 2 illustrates a block diagram for an example method 200 for preparing some embodiments of an energy composition. The blocks shown in FIG. 2 can be rearranged or removed from method 200. Additional blocks, steps, process, and/or methods, etc. may be added to method 200.

Method 200 may include block 201, block 203, block 205, block 207, block 209, and block 211. Example embodiments may include mixing, which may include, for example, mixing through a high sheer mixer or another type of mixer. The high sheer mixer may include, for example, any method for substantially mixing two or more components, such as, for example, a high speed blender, a miller, a grinder, and/or any other method suitable for substantially mixing two or more ingredients.

Block 201 may include preparing one or more herbs to be included in the energy composition. Herbs that include teas are not expressly limited to use with water, but may also include, for example, waterless compositions such as powders, leaves, and/or any other compositions that do not substantially use water. Preparing the herbs may include, for example, altering the state of the herb in a manner suitable for mixing with one or more of the delivery system, active ingredient, methylxanthine, gumming agent, preservative, coloring, and/or flavorant, etc. For example, such preparation may include shredding, crushing, dicing, burning, pulverizing, and/or any other manner suitable for mixing with one or more of the delivery system, active ingredient, methylated xanthine, gumming agent, preservative, coloring, and/or flavorant, etc. Preparing the herbs may include any method for extracting components from the herbs into a form suitable for mixing with one or more of the delivery system, active ingredient, methylxanthine, gumming agent, preservative, coloring, and/or flavorant. Such preparing may include forms such as, for example, steeping, brewing, boiling, steaming, mixing, and/or any other preparation method suitable for extracting chemicals from herbs. The herb may be prepared in a manner and for a time such that substantially all and/or some components of the herb, or herbs, may be extracted.

In some embodiments, the preparation may be done in a manner suitable for increasing the bio-availability of the extracted components. In some embodiments, the components of the herb may be held naturally in a bound matrix, and the preparation may be done in a manner suitable for releasing the extracted components from the bound matrix. Increasing the bio-availability of the extracted components may increase the rate at which the energy composition provides the perceived effects. Such preparation may include, for example, brewing the herb, or herbs, for a suitable time and in a suitable temperature. In some embodiments, the preparation may include, for example, using only the extracted components. In some embodiments, the herb and extracted components may be used.

In some embodiments, preparation of the herb may include, for example, heating water to a temperature between 80 and 100 degrees Celsius, adding the Mormon tea to the water to create an herb and water mixture, removing the herb and water mixture from the heat, and soaking the herb and water mixture for no shorter than 30 minutes and/or until the active constituents are released. In some embodiments, the water, for example, is heated to at least 80 degrees, but the water is not brought to a boil.

In some embodiments, a stainless steel mesh basket, for example, may be used to add the herb to the water. For example, the stainless steel mesh basket may include mesh of about 5 μm. Filters may also be used. However, in a large production, filters may cause possible problems with allowing water to completely saturate the herb. Using the stainless steel mesh basket, for example, may allow water to substantially or completely saturate the herb.

Brewing the herb may create a more effective energy composition. For example, brewing the herb may, for example, eliminate sedimentation. Brewing the herb may also provide a more immediate effect of the energy composition. Additionally, the effect of the herb strengthens relative to the length of time the herb is brewed.

Block 203 may include, for example, hydrating the gumming agent. Hydration may include any method for substantially hydrating the gumming agent to about 10 parts water to 1 part gumming agent. Some embodiments may include, for example, mixing the gumming agent with water. Hydration of the gumming agent may include various mixing times to mix the gumming agent with water. Some embodiments may include a hydration time, for example, of about seven to ten minutes.

Block 205 may include mixing the prepared herbs with the hydrated gumming agent. Block 207 may include mixing the preservatives with the mixed herbs and hydrated gumming agent. Block 209 may include mixing the active ingredients with the mixed herbs, hydrated gumming agent, and preservatives. In some embodiments, the order may include mixing active ingredients with a lower dissolvability before mixing active ingredients with a higher dissolvability. For example, block 209 may include mixing taurine before other active ingredients. Block 211 may include mixing the flavorant and/or flavorants with the mixed herbs, hydrated gumming agent, preservatives, and active ingredients. In some embodiments, the order of mixing blocks 205, 207, 209, and 211 may vary. For example, the active ingredients may be mixed with the prepared herb before the hydrated gumming agent and/or preservatives are added to the solution.

Embodiments of the method 200 that include mixing may include various mixing times configured to combine the components of the energy composition into a suitable form that may provide the perceived effects of an energy composition.

Modifications may be made to method 200 without changing the scope of the disclosure. In some embodiments, block 203 may include, for example, hydrating the gumming agent with the preservatives. Method 200 may also omit any block in which the corresponding component is not included in the composition. For example, embodiments omitting the gumming agent may omit block 203; embodiments omitting active ingredients may omit block 209.

Each component used herein may have a different name associated with such a component. For example, Mormon tea may be referred to as ephedra viridis or Brigham tea. Ephedra viridis is a species of the ephedra genus. There may be up to 36 different plant species of ephedra, many of which may include weight loss or fat burning properties.

In some embodiments, Ephedra viridis, or Mormon tea, does not contain ephedrine alkaloids such as, for example, ephedrine alkaloids that have been banned by the FDA. Ephedra viridis may include thermogenic properties and energy inducing properties and without, for example, the undesired side effects caused by ephedra sinica.

The addition of theobromine manages the bioavailability of caffeine. Theobromine is a metabolite of caffeine. The addition of theobromine may maintain the alkaloid effect of the energy composition for a longer period of time.

In some embodiments, an active constituent is not the same as active ingredient. Active constituents, for example, may be released when an herb is heated. IN addition, active constituents, for example, may be released from the herb that may help increase the perceived effects. An active ingredient, for example, may be a separate ingredient that may be added to the energy composition.

The term “substantially” means within 5% or 10% of the value referred to or within manufacturing tolerances.

Various embodiments are disclosed. The various embodiments may be partially or completely combined to produce other embodiments.

All examples and conditional language recited herein are intended for pedagogical objects to aid the reader in understanding the present invention and the concepts contributed by the inventor to furthering the art, and are to be construed as being without limitation to such specifically recited examples and conditions. Although embodiments of the present invention have been described in detail, it should be understood that the various changes, substitutions, and alterations may be made hereto without departing from the spirit and scope of the present invention.

What is claimed is:

1. A method of formulating an energy composition, comprising:
heating water to a temperature from 80 to 100 degrees Celsius;
adding Mormon tea in an amount from about 0.000082 g/mL to about 0.0047 g/mL to the water, to create an herb and water mixture;
removing the herb and water mixture from the heat;
soaking the herb and water mixture for no shorter than 30 minutes or until active constituents are released;
mixing the herb and water mixture, a methylxanthine including theobromine in an amount from about 0.000083 g/mL to about 0.012 g/mL, and an active ingredient including phenylethylamine HCl in an amount from about 0.00094 g/mL to about 0.010 g/mL; and
formulating the energy composition in a predetermined volume for consumption.

2. The method of formulating an energy composition in claim 1, wherein the methylxanthine includes caffeine in an amount from about 0.0028 g/mL to about 0.0050 g/mL.

3. The method of formulating an energy composition in claim 2, wherein the methylxanthine includes caffeine and theobromine, coupled to stabilize the perceived effects, in an amount from about 0.0028 g/mL to about 0.0062 g/mL.

4. The method of formulating an energy composition in claim 1, wherein malic acid is present in an amount from about 0.010 g/mL to about 0.014 g/mL.

5. The method of formulating an energy composition in claim 1, wherein yohimbine HCl is present in an amount from about 0.000049 g/mL to 0.000098 g/mL.

6. An energy composition comprising:
Mormon tea in an amount from about 0.00037 g/mL to about 0.0015 g/mL;
theobromine and caffeine in an amount from about 0.0031 g/mL to about 0.0039 g/mL; and
phenylethylamine HCl in an amount from about 0.0018 g/mL to about 0.0042 g/mL.

7. The energy composition in claim 6, \wherein water is added and the Mormon tea is brewed in the water for no shorter than 30 minutes or until active constituents are released.

8. The energy composition in claim 6, wherein caffeine is present in an amount from about 0.0030 g/mL to about 0.0038 g/mL.

9. The energy composition in claim 6, wherein malic acid is present in an amount from about 0.010 g/mL to about 0.014 g/mL.

10. The energy composition in claim 6, wherein yohimbine HCl is present in an amount from about 0.000049 g/mL to 0.000097 g/mL.

11. An energy composition comprising:
an herb including Mormon tea in an amount from about 0.000082 g/mL to about 0.0047 g/mL;
a methylxanthine including theobromine in an amount from about 0.000083 g/mL to about 0.0012 g/mL; and
an active ingredient including phenylethylamine HCl in an amount from about 0.00094 g/mL to about 0.0083 g/mL.

12. The energy composition in claim 11, further comprising water and wherein the Mormon tea is brewed in water for no shorter than 30 minutes or until active constituents are released.

13. The energy composition in claim 11, wherein the herb includes guayusa in an amount from about 0.00041 g/mL to about 0.012 g/mL.

14. The energy composition in claim 11, wherein the methylxanthine includes caffeine in an amount from about 0.0028 g/mL to about 0.0050 g/mL.

15. The energy composition in claim 11, wherein the methylxanthine includes caffeine and theobromine, coupled to stabilize the perceived effects, in an amount from about 0.0028 g/mL to about 0.0062 g/mL.

16. The energy composition in claim 11, wherein the active ingredient further comprises malic acid in an amount from about 0.011 g/mL to about 0.014 g/mL.

17. The energy composition in claim 11, wherein the active ingredient further comprises yohimbine HCl in an amount from about 0.000066 g/mL to 0.000083 g/mL.

18. An energy composition comprising:
Mormon tea in an amount from about 0.000018 g/mL to about 0.019 g/mL;
theobromine in an amount from about 0.000060 g/mL to about 0.0019 g/mL;
caffeine in an amount from about 0.00083 g/mL to about 0.012 g/mL; and
an active ingredient including phenylethylamine HCl in an amount from about 0.000018 g/mL to about 0.010 g/mL.

19. The energy composition in claim 18, further comprising water and wherein the Mormon tea is brewed in water for no shorter than 30 minutes or until active constituents are released.

20. The energy composition in claim 18, wherein the active ingredient further comprises malic acid in an amount from about 0.0083 g/mL to about 0.034 g/mL.

21. The energy composition in claim 18, wherein the active ingredient further comprises yohimbine HCl in an amount from about 0.000015 g/mL to 0.00017 g/mL.
